(19) United States
(12) Patent Application Publication
(10) Pub. No.: US 2025/0057699 A1
(43) Pub. Date: Feb. 20, 2025

(54) DISPOSABLE ABSORBENT GARMENT FOR CARE OF WOUND PRODUCING FLUID LEAKAGE

(71) Applicant: JM Wound Care LLC, Conroe, TX (US)

(72) Inventor: Taquiela Delisa Wright, Conroe, TX (US)

(21) Appl. No.: 18/674,503

(22) Filed: May 24, 2024

Related U.S. Application Data
(60) Provisional application No. 63/469,308, filed on May 26, 2023.

(51) Int. Cl. A61F 13/00

(52) U.S. Cl. CPC A61F 13/00063; A61F 2013/0028

(57) ABSTRACT

A disposable wound care garment with multiple layers may cover a wound on the epidermis and wick away and store leakage of fluids from the wound. The wound garment may include a medication temporary storage and dispensing region to dispense medication to the wound on the epidermis, while also wicking away fluid. The wound garment may be used on extremities, such as, for example, the legs or arms. The wound care garment may be a component of a treatment plan for conditions, such as, for example, lymphedema, primary lymphedema, cellulitis, congestive heart failure, renal failure, and chronic venous insufficiency.

### CROSS-REFERENCE TO RELATED APPLICATIONS

This application is related and claims priority to U.S. Provisional Application 63/469,308 filed May 26, 2023 and titled DISPOSABLE ABSORBENT GARMENT FOR CARE OF WOUND PRODUCING FLUID LEAKAGE, which is incorporated by reference in entirety.

### FIELD OF THE INVENTION

The disclosure relates to disposable absorbent garments and, particularly, disposable absorbent garments for use on wounds and the epidermis.

### BACKGROUND OF THE INVENTION

There are more than 200,000 U.S. cases per year for lymphedema, which leads to lower-limb swelling and fluid leakage. This may be caused by renal disease, cancer, drug therapy, and heart failure. Primary lymphedema is caused by a blood clot in the deep veins of the leg, and edema may also be caused by a tumor blocking the flow of blood or another fluid called lymph. This condition is critical and may lead to life-threatening infections, or other critical illnesses.

Cellulitis is another common condition, with more than 200,000 U.S. cases per year. It occurs when bacteria, most commonly streptococcus and staphylococcus, enter through a crack or break in the skin. The condition may lead to the affects skin to become red, swollen, warm, and sore. An open sore may leak fluid.

Congestive heart failure, a common condition in the US, causes fluid to back up in the veins and capillaries in the body. This backup can cause pressure on the capillaries and fluid may seep out into the tissue causing swelling and even “weeping” of tissue, especially in the legs and feet.

Another common condition, renal failure, is caused by damage to the kidneys, and can cause decreased levels of protein in the blood. This can contribute to capillary leaking and swelling. The edema associated with kidney disease usually occurs in the legs and around the eyes. Many patients wait until symptoms are well advanced before seeking medical help, complicating the condition even further.

A very common condition, chronic venous insufficiency (CVI) is a condition that occurs when the venous wall and/or valves in the leg veins do not work effectively, making it difficult for blood to return to the heart from the legs. CVI causes blood to “pool” or collect in these veins, and this pooling is called stasis.

All of conditions mentioned may lead to fluid leakage from a wound on the body. Other conditions may also lead to fluid leakage from a wound on the body. There is need for improved disposable garments to cover and protect a wound area or other source of fluid leakage from the skin (collectively, a wound area or other source of fluid leakage from the skin being a “wound”). There is need to remove fluid leakage produced from a wound, or produced from a source of fluid leakage from the skin, away from the wound and skin to prevent worsening of the wound or condition, and prevent development of related conditions.

The present disclosure relates to improvements of disposable garments that may be used to cover and protect a wound producing fluid leakage or a source producing fluid leakage from the skin.

### BRIEF SUMMARY OF THE INVENTION

Existing wound garments suffer problems including poor or limited capacity to remove or carry away, such as by wicking away, excessive amounts of fluid produced from a wound that has leakage. Existing wound garments also suffer problems including poor or limited capacity to receive and store or hold for disposal, the excessive amounts of fluid produced from a wound that has leakage, or even that portion of fluid which can be collected, removed and carried away from the wound or source of fluid leakage. This may lead to the leaking fluid gathering on the skin and causing other infections or worsening of the existing condition. Furthermore, fluid leakage which exceeds the capacity of a disposable garment to capture, remove, and store or hold for disposal, may leak onto the patient's surroundings such as bedding, furniture, and apparel. Fluid leakage which exceeds the capacity of a disposable garment to capture, remove and receive, store or hold for disposal also requires increased labor for a caregiver to repeatedly remove and replace disposable garments having insufficient capacity to remove and store fluid leakage.

The above-mentioned shortcomings, disadvantages and problems are addressed herein, as will be understood by those skilled in the art upon reading and studying the following specification. This summary is provided to introduce a selection of concepts in simplified form that are further described below in more detail in the detailed description. This summary is not intended to identify key or essential features of the claimed subject matter.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Disclosed subject matter includes a disposable garment to cover a wound producing fluid leakage, wherein the garment may include multiple layers. The garment layers may include at least one outer storage layer, an inner absorption layer, an inner medication temporary storage layer, and an inner dispensing layer.

Disclosed subject matter includes a disposable garment that may include at least one compression layer. The at least one compression layer may include, for example, a first compression layer to provide a first structural function and a second compression layer to provide a second structural function.

Disclosed subject matter includes a disposable garment that may include a treatment fluid introduction path to load treatment fluid into the inner medication temporary storage layer without obstructing or loading the inner absorption layer.

Apparatus, systems, and methods of varying scope are described herein. These aspects are indicative of various non-limiting ways in which the disclosed subject matter may be utilized, all of which are intended to be within the scope of the disclosed subject matter. In addition to the aspects and advantages described in this summary, further aspects, features, and advantages will become apparent by reference to the associated drawings, detailed description, and claims.

### BRIEF DESCRIPTION OF THE DRAWINGS

The novel features believed characteristic of the disclosed subject matter will be set forth in any claims that are filed later. The disclosed subject matter itself, however, as well as a preferred mode of use, further objectives, and advantages thereof, will best be understood by reference to the following detailed description of an illustrative embodiment when read in conjunction with the accompanying drawings, wherein:

FIG. 1 is a simplified deconstructed view showing the layers of a disposable wound garment, in an embodiment.

FIG. 2 is a simplified partial cross-sectional view, illustrating combined layers of a disposable wound garment as shown generally in FIG. 1.

FIG. 3 is a simplified perspective view showing a disposable wound garment used to cover a wound on an extremity, in an embodiment.

FIG. 4 is a simplified diagram, illustrating the application to a patient's arm of a disposable wound garment as shown generally in FIG. 3.

### DETAILED DESCRIPTION OF ILLUSTRATIVE EMBODIMENTS

In this detailed description, reference is made to the accompanying drawings which form a part hereof, and in which is shown by way of illustration specific embodiments which may be practiced. Reference now should be made to the drawings, in which the same reference numbers are used throughout the different figures to designate the same components. These embodiments are described in sufficient detail to enable those skilled in the art to practice the embodiments and disclosure. It is to be understood that other embodiments may be utilized, and that logical, mechanical, electrical, and other changes may be made without departing from the scope of the embodiments and disclosure. In view of the foregoing, the following detailed description is not to be taken as limiting the scope of the embodiments or disclosure.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be appreciated that for simplicity and clarity of illustration, where considered appropriate, reference numerals may be repeated among the figures to indicate corresponding or analogous elements. In addition, numerous specific details are set forth in order to provide a thorough understanding of the implementations described herein. However, it will be understood by those of ordinary skill in the art that the implementations described herein may be practiced without these specific details. In other instances, well-known methods, procedures and components have not been described in detail so as not to obscure the implementations described herein. Also, the description is not to be considered as limiting the scope of the implementations described herein.

The detailed description set forth herein in connection with the appended drawings is intended as a description of exemplary embodiments in which the presently disclosed apparatus and system can be practiced. The term “exemplary” used throughout this description means “serving as an example, instance, or illustration,” and should not necessarily be construed as preferred or advantageous over other embodiments.

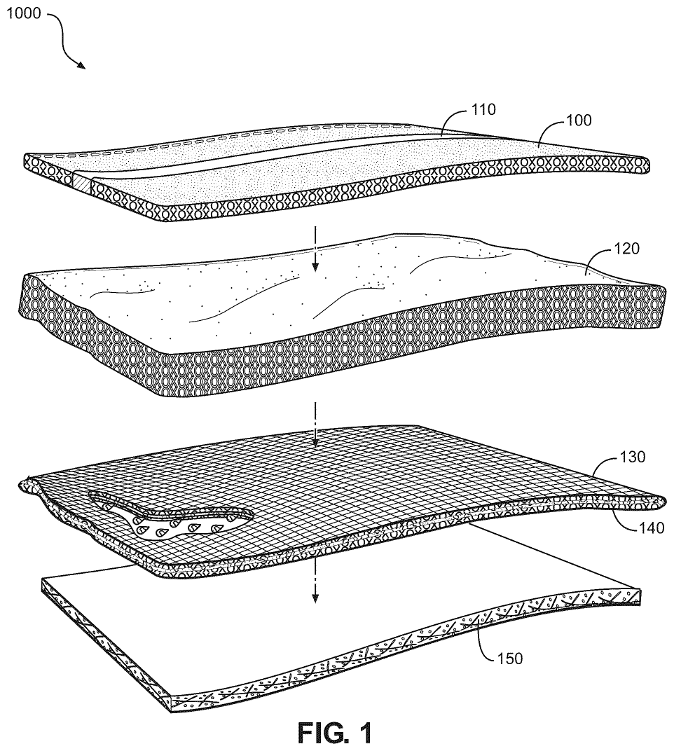

Illustrated in FIG. 1 is a simplified deconstructed diagram of a wound garment 1000, illustrating its layers. The wound care garment may comprise at least one outer storage layer 120 located apart from the wound, said outer storage layer may receive and store biological fluid wicked away from the wound. Said wound garment 1000 may further comprise an inner absorption layer 130, said inner absorption layer 130 may wick away biological fluid from the wound on the epidermis 150 to the outer storage layer 120 located apart from the wound on the epidermis 150. Said wound garment 1000 may further comprise an inner medication temporary storage and dispensing layer 140, said inner medication temporary storage and dispensing layer 140 may contain a treatment fluid comprising a medication component to treat a condition or property of the wound on the epidermis 150. Said inner medication temporary storage and dispensing layer 140 may dispense to the wound area on the epidermis 150 said treatment fluid comprising a medication component to treat a condition or property of the wound. Said wound care garment 1000 may further comprise a first compression layer 100, which may provide at least very light pressure to draw the garment against the wound on the epidermis 150. The first compression layer 100 may further comprise a copper lining 110, which may increase stability. The wound garment 1000 may further comprise the compression layer 100 being a second compression layer 100 which may provide higher pressure to more firmly support the high volume fluid storage region in relation to the wearer, but may direct this pressure on the epidermis 150 apart from the wound area being treated. Said second compression layer 100 may further comprise copper lining 110 for increased stability.

The inner medication temporary storage and dispensing layer 140 and inner absorption layer 130 may be exposed to nonoverlapping regions of the surface area of the wound on the epidermis 150, and may avoid said wicking and dispensing in the same region of surface area of the wound on the epidermis 150.

The absorption layer 130 may be exposed to a nonoverlapping region of the surface area of the wound on the epidermis 150 in relation to the inner medication temporary storage and dispensing layer 140, to avoid said wicking flow of biological fluid through the inner medication temporary storage and dispensing layer 140.

The absorption layer 130 may be exposed to a nonoverlapping region of the surface area of the wound on the epidermis 150 in relation to the inner medication temporary storage and dispensing layer 140, to avoid wicking flow of biological fluid through the inner medication temporary storage and dispensing layer 140.

The outer storage layer 120 may be removable and replaceable.

The wound garment 1000 may further comprise a treatment fluid introduction path to load treatment fluid into the inner medication temporary storage and dispensing layer without obstructing or loading the inner absorption layer 130.

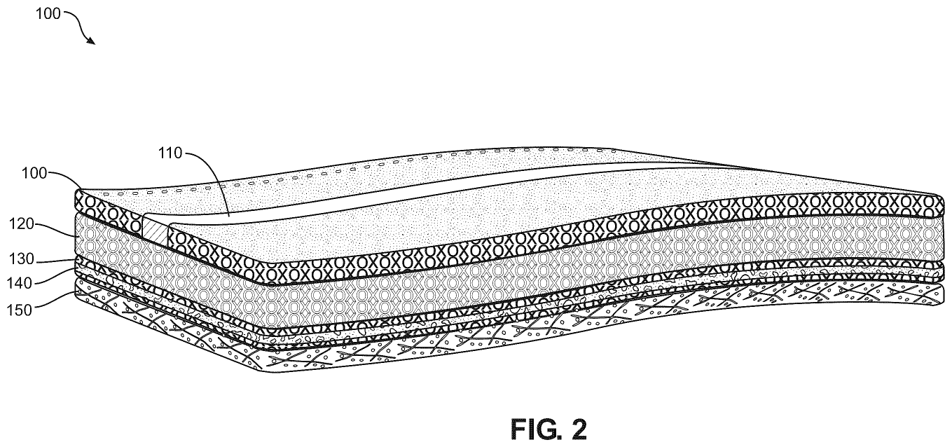

Illustrated in FIG. 2 is the wound garment 1000 as shown generally in FIG. 1, constructed of, and including, the plurality of layers 100, 110, 120, 130, 140, and 150. The plurality of layers may be ordered as illustrated in FIG. 2. The plurality of layers may be joined in any suitable manner, such as by stitching which penetrates the plurality of ordered layers or a subset of the ordered layers, adhesives between abutting layers, and/or releasable fasteners such as, for example, hook and loop type fasteners of the Velcro® type adjoining abutting layers.

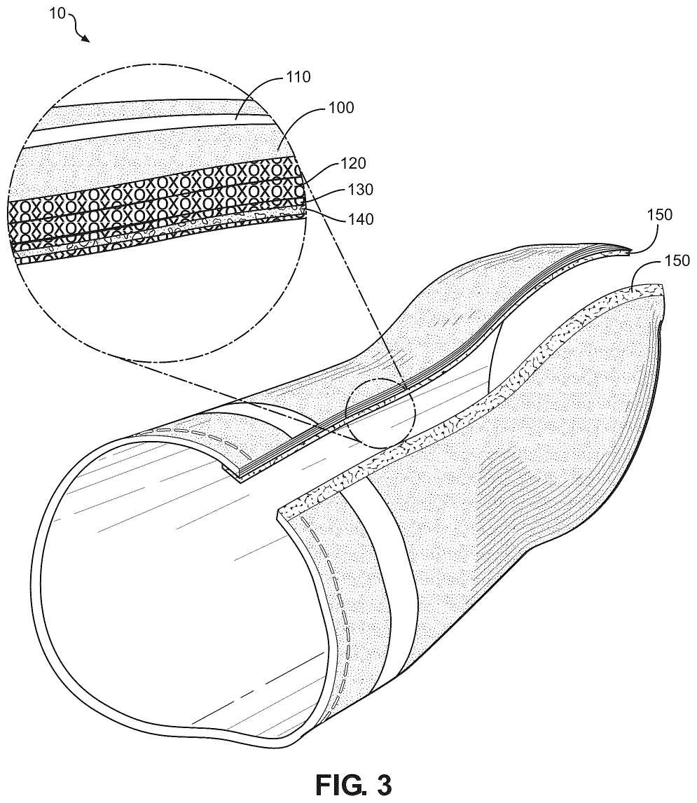

Illustrated in FIG. 3 is the wound garment 10 formed into a cylindrical shape to be used for wrapping around an extremity of a patient and pressed against the epidermis 150. The wound garment 10 may contain embodiments 100, 110, 120, 130, 140.′

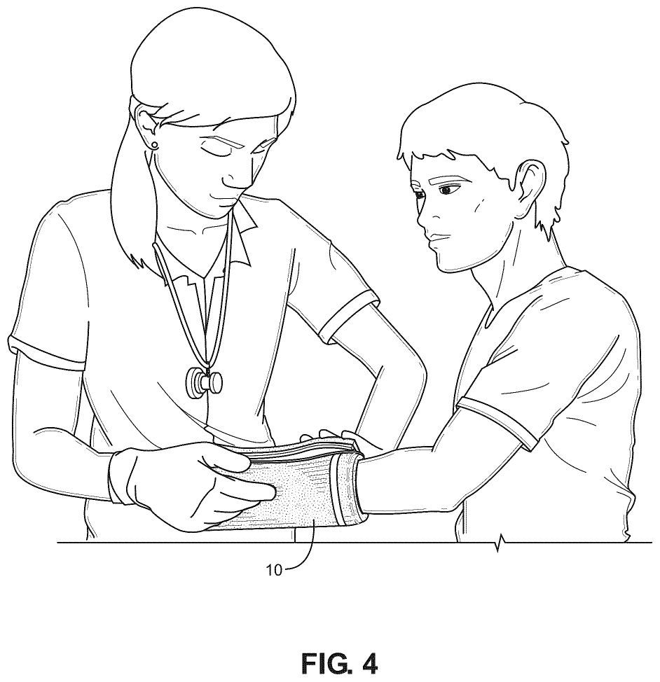

Illustrated in FIG. 4 is a installation of the wound garment 10 to a patient's arm. The wound garment 10 may also be applied to any other extremity of a patient, such as, for example, the leg. The wound garment 10 may also, for example, be self-installed, or installed by a medical professional or non-medical professional or caregiver.

One or more embodiments may comprise materials made from wood cellulose fiber and/or polyacrylate. One or more embodiments may comprise materials made from polyethylene, and an absorbent rayon/cellulose blend. One or more embodiments may comprise materials made from polyhexamethylene and biguanide (PHMB). One or more embodiments may comprise materials made from cotton, rubber, nylon, polyester, and/or spandex. One or more embodiments may comprise materials made from polyhexamethylene biguanide (PHMB), benzalkonium chloride and/or polyhexamethylene. One or more embodiments may comprise materials made from hydrocolloid dressing. One or more embodiments may comprise materials made from methacrylate. One or more embodiments may comprise materials made from polyvinyl pyrrolidine. One or more embodiments may comprise materials made from silicone-based rubber foam. One or more embodiments may comprise materials made from wick fabric. One or more embodiments may comprise materials made from stretch nylon. One or more embodiments may comprise materials made from cotton. One or more embodiments may comprise materials made from bamboo. One or more embodiments may comprise materials made from a nonstick adhesive. One or more embodiments may comprise materials made from a non-adhesive padding. One or more embodiments may comprise materials made from rubber. One or more embodiments may comprise materials made from hydrophilic fabric. One or more embodiments may comprise materials made from a CBD patch.

Apparatus, methods, and systems according to embodiments of the disclosure are described. Although specific embodiments are illustrated and described herein, it will be appreciated by those of ordinary skill in the art that any arrangement which is calculated to achieve the same purposes can be substituted for the specific embodiments shown. This application is intended to cover any adaptations or variations of the embodiments and disclosure. For example, although described in terminology and terms common to the field of art, exemplary embodiments, systems, methods and apparatus described herein, one of ordinary skill in the art will appreciate that implementations can be made for other fields of art, systems, apparatus or methods that provide the required functions. The invention should therefore not be limited by the above-described embodiments, methods, and examples, but by all embodiments and methods within the scope and spirit of the invention.

In particular, one of ordinary skill in the art will readily appreciate that the names of the methods and apparatus are not intended to limit embodiments or the disclosure. Furthermore, additional methods, steps, and apparatus can be added to the components, functions can be rearranged among the components, and new components to correspond to future enhancements and physical devices used in embodiments can be introduced without departing from the scope of embodiments and the disclosure. One of skill in the art will readily recognize that embodiments are applicable to future systems, future apparatus, future methods, and different materials.

All methods described herein can be performed in a suitable order unless otherwise indicated herein or otherwise clearly contradicted by context. The use of any and all examples, or exemplary language (e.g., “such as”), is intended merely to better illustrate the disclosure and does not pose a limitation on the scope of the disclosure unless otherwise claimed. No language in the specification should be construed as indicating any non-claimed element as essential to the practice of the disclosure as used herein. Terminology used in the present disclosure is intended to include all environments and alternate technologies that provide the same functionality described herein.

What is claimed is:

1. A disposable wound care garment comprising:
(a) at least one outer storage layer located apart from the wound, said outer storage layer receives and stores biological fluid wicked away from the wound;
(b) an inner absorption layer, said inner absorption layer wicks away biological fluid from the wound to the (a) outer storage layer located apart from the wound;
(c) an inner medication temporary storage layer, said inner medication temporary storage layer contains a treatment fluid comprising a medication component to treat a condition or property of the wound;
(d) an inner dispensing layer, said inner dispensing layer dispenses to the wound area said treatment fluid comprising a medication component to treat a condition or property of the wound.

2. The disposable wound care garment of claim 1, further comprising:
a first compression layer providing at least very light pressure to draw the garment against the wound.

3. The disposable wound care garment of claim 2, further comprising:
the first compression layer comprising copper lining to increase stability.

4. The disposable wound garment of claim 1, further comprising:
a second compression layer providing higher pressure to more firmly support the high volume fluid storage region in relation to the wearer, but directing this pressure on flesh apart from the wound area being treated.

5. The disposable wound garment of claim 4, further comprising:
the second compression layer comprising copper lining for increased stability.

6. The disposable wound garment of claim 1, further comprising:
the inner dispensing layer and inner absorption layer exposed to nonoverlapping regions of the surface area of the wound, thus avoiding said wicking and dispensing in the same region of surface area of the wound.

7. The disposable wound garment of claim 1, further comprising:
the absorption layer is exposed to a nonoverlapping region of the surface area of the wound in relation to the inner medication temporary storage layer, to avoid said wicking flow of biological fluid through the inner medication temporary storage layer.

8. The disposable wound garment of claim 1, further comprising:
the absorption layer is exposed to a nonoverlapping region of the surface area of the wound in relation to the inner medication dispensing layer, to avoid wicking flow of biological fluid through the inner medication dispensing layer.

9. The disposable wound garment of claim 1, further comprising:
the outer storage layer is removable and replaceable.

10. The disposable wound garment of claim 1, further comprising:
a treatment fluid introduction path to load treatment fluid into the inner medication temporary storage layer without obstructing or loading the inner absorption layer.
